(19) United States
(12) Patent Application Publication
(10) Pub. No.: US 2020/0346224 A1
(43) Pub. Date: Nov. 5, 2020

(54) MINERAL BENEFICIATION METHOD USING BIOREAGENT EXTRACTED FROM GRAM POSITIVE BACTERIA

(71) Applicant: VALE S.A., Rio de Janeiro RJ (BR)

(72) Inventors: Maurício Leonardo TOREM, Rio de Janeiro (BR); Jhonatan Gerardo Soto PUELLES, Cusco (PE); Antonio Gutiérrez MERMA, Rio De Janeiro (BR); Carlos Alberto Castañeda OLIVERA, Rio De Janeiro (BR); Lisa Marinho do ROSÁRIO, Rio de Janeiro (BR); Flávia Paulucci Cianga SILVAS, Ouro Preto (BR)

(73) Assignee: VALE S.A., Rio de Janeiro, RJ (BR)

(21) Appl. No.: 16/614,321

(22) PCT Filed: May 16, 2018
(86) PCT No.: PCT/BR2018/050158

§ 371 Date: Jul. 9, 2020

Related U.S. Application Data
(60) Provisional application No. 62/507,028, filed on May 16, 2017.

(51) Int. Cl. B03D 1/016; C12N 1/20; C01G 49/04; C01F 11/18; C01B 25/32; C22B 3/18

(52) U.S. Cl. CPC B03D 1/016; C12N 1/20; C01G 49/04; C01F 11/185; B03D 2203/04; C22B 3/18; B03D 2201/06; B03D 2201/04; B03D 2201/02; C01B 25/327

(57) ABSTRACT

The object of this invention is to provide a method of mineral flotation using bioreagents extracted from Gram positive bacteria Rhodococcus opacus and Rhodococcus erythropolis. In this sense, mineral floatability was evaluated using bioreagent extracted from Gram positive bacteria to determine its potential as an alternative to synthetic reagents and also an alternative to the use of microorganisms themselves (biomass).

### CROSS REFERENCE TO RELATED APPLICATIONS

This application is a 35 U.S.C. § 371 National Phase of PCT Application No. PCT/BR2018/050158 filed May 16, 2018, entitled “MINERAL BENEFICIATION METHOD USING BIOREAGENT EXTRACTED FROM GRAM POSITIVE BACTERIA,” which claims benefit to U.S. Provisional Application No. 62/507,028 filed May 16, 2017. The disclosure of these prior applications are hereby incorporated by reference herein in their entirety

### INVENTION FIELD

This invention is primarily intended for the mining industry, and comprises a method of mineral beneficiation using bioreagent extracted from Gram positive bacteria (Rhodococcus opacus and Rhodococcus erythropolis).

### BACKGROUND OF THE INVENTION

One of the main processes of mineral concentration used by the mining industry is froth flotation. Bioflotation is defined as a separation process in which the mineral of interest is selectively floated or depressed using reagents of biological origin, known as bioreagents.

Bioflotation has been extensively studied in recent years as an attractive alternative to replace conventional reagents with those environmentally friendly. Bioreagents are characterized by low toxicity and ease of degradation when disposed of and the raw material for their production is low cost, renewable and easily available. In addition, bioreagents can be used in the processing of low content ore and mining tailings, making it possible to exploit economically impracticable deposits.

On the other hand, even though there is evidence that bioflotation is a process that presents good recovery and selectivity, there are factors that inhibit the development of the technique, including small technological advance and little knowledge of the mechanisms, kinetics and thermodynamics of the process.

Bioreagents are a heterogeneous mixture of various compounds that are difficult to characterize, making it difficult to understand the specific mechanisms involved in the froth flotation process, where bioreagents are able to selectively modify the surface of the mineral concerned. In addition, it is important to note that the theoretical models used to describe the behavior of mineral/bacterial adhesion do not consider biological factors. The inclusion of these factors is of great importance for a complete understanding of the processes that occur during bioflotation.

The use of microorganisms and/or their metabolic products as reagents, in particular, collectors, foaming reagents and modifiers in mineral processing operations, has become very attractive because it has great technological potential, is environmentally acceptable, and presents selectivity in mineral particle processing. These microorganisms and/or their metabolic products can modify the mineral surface, either directly or indirectly. The direct mechanism involves the direct adhesion of microbial cells to mineral particles, while the indirect mechanism refers to metabolism products or soluble cell fractions that act as active reagents in surface. Both interactions lead to changes in surface chemistry, making it hydrophilic or hydrophobic depending on the character of the bioreagent and mineral concerned.

The major function of microorganisms and/or their metabolic products as bioreagent in mineral processing is related to the presence of nonpolar functional groups (hydrocarbon chains) and polar groups (carboxyls, phosphates, hydroxyls) on their cell surface or in the intra and/or extracellular compounds produced by microorganisms, which can modify the interface properties and thereby change the amphipathic characteristics of a mineral surface.

Rhodococcus erythropolis and Rhodococcus opacus bacteria are Gram positive, non-pathogenic and are found widely in nature from a wide variety of sources.

The document CN102489415, for example, describes the use of Rhodococcus erythropolis bacteria as a collecting agent in a froth flotation process of a system containing hematite. This document differs from the present invention by the fact it uses as a collecting agent the bacterium itself (biomass), not a bioreagent extracted from a bacterium.

The document CN102911904 describes the use of bacteria as collecting agents in an ore flotation process containing refractory hematite. As in CN102489415, this document differs from the present invention by the fact it uses as a collecting agent the bacterium itself (biomass), not a bioreagent extracted from a bacterium.

The article “Biosurfactant production by Rhodococcus Erythropolis and its application to oil removal”, published on Oct. 29, 2010 by the Universidade Federal do Rio de Janeiro, mentions a biosurfactant extracted from the bacterium Rhodococcus erythropolis used to treat oil-contaminated soil. This invention differs from said document because it is a mineral flotation, not oil-contaminated soil treatment.

The article “Flocculation and flotation response of Rhodococcus erythropolis to pure minerals in hematite ores”, published on Feb. 27, 2013 by the University of Science & Technology Beijing, describes the use of bacterium Rhodococcus erythropolis as a collecting agent in a process of flotation of a system containing hematite. As in CN102489415 and CN102911904, this document differs from the present invention by the fact it uses as a collecting agent the bacterium itself (biomass), not a bioreagent extracted from a bacterium.

The article “Flotation of serpentinite and quartz using biosurfactants”, published May 6, 2012 by Wroclaw University of Technology (Poland), mentions a biosurfactant extracted from the bacteria Bacillus circulars and Streptomyces sp. used in quartz and serpentine flotation. This invention differs from said document because they are different bacteria, as well as different minerals to be recovered by froth flotation process.

As will be further detailed below, this invention provides a method of mineral beneficiation using bioreagents extracted from the bacteria Rhodococcus opacus and Rhodococcus erythropolis.

### SUMMARY OF THE INVENTION

The main object of this invention is to provide a method of mineral beneficiation using bioreagents extracted from Rhodococcus opacus and Rhodococcus erythropolis bacteria.

Thus, the process of extracting the metabolites, especially protein compounds, from the bacteria Rhodococcus opacus and Rhodococcus erythropolis was evaluated in order to use them as collecting bioreagents in mineral flotation, since proteins tend to provide hydrophobic character on mineral surfaces, thus favoring the flotation process.

In this sense, mineral floatability was evaluated using bioreagent extracted from bacteria of the genus Rhodococcus to determine its potential as an alternative to synthetic reagents and also an alternative to the use of microorganisms themselves (biomass).

### BRIEF DESCRIPTION OF THE FIGURES

The detailed description given below refers to the figures and their respective reference numerals:

FIG. 1 illustrates a process flowchart for extracting bioreagent from microorganisms;

FIG. 2 shows an infrared spectrum of bacterium R. opacus (blue line) and crude bioreagent (black line).

FIG. 3 shows an infrared spectrum of bacterium R. erythropolis (blue line) and crude bioreagent (black line);

FIG. 4 is a graph illustrating the effect of bioreagent concentration on surface tension of deionized water at 20° C. and neutral pH: continuous line, bioreagent extracted from R. opacus bacteria and bioreagent dotted line extracted from R. erythropolis bacteria;

FIG. 5 presents bar diagrams comparing hematite floatability using bacteria (biomass) and bioreagent: (a) pH3, (b) pH5, (c) pH7, (d) pH9, (e) pH11;

FIG. 6 is a graph illustrating the floatability of hematite at different concentrations of bioreagent extracted from the R. opacus bacterium;

FIG. 7 is a graph illustrating the floatability of hematite at different concentrations of bioreagent extracted from the R. erythropolis bacterium;

FIG. 8 presents bar diagrams comparing the floatability of hematite, quartz, dolomite, calcite and apatite using bioreagent extracted from R. opacus bacteria: (a) pH3, (b) pH5, (c) pH7, (d) pH9, (e) pH11;

FIG. 9 presents bar diagrams comparing the floatability of hematite, quartz, dolomite, calcite and apatite using bioreagent extracted from R. erythropolis bacteria: (a) pH3, (b) pH5, (c) pH7, (d) pH9, (e) pH11.

### DETAILED DESCRIPTION OF THE INVENTION

Preliminarily, it is emphasized that the following description will start from a preferred embodiment of the invention. As will be apparent to any person specialized in the subject, however, the invention is not limited to that particular embodiment.

This invention is a method of mineral beneficiation using bioreagents extracted from the bacteria Rhodococcus opacus and Rhodococcus erythropolis, said method comprising the phases of i) comminuting the ore and preparing the pulp; ii) addition of reagent and conditioning; ii) flotation.

As widely known by technicians in the field, growth broths used for inoculating bacteria in the present invention should preferably contain sources of nutrients, proteins and carbohydrates. Broths may be prepared using commercial reagents or there may be partial or total substitution with ingredients from other production chains, for example, food industry residue. The growth of microorganisms may occur in a rotary kiln or, for large scale processes, fermenters or bioreactors may be used. The temperature and the presence of contaminants should be controlled.

In accordance with the present invention, the extraction of bioreagent from Rhodococcus bacteria (opacus, erythropolis) is carried out by a solvent extraction process, preferably hot ethanol extraction (100-140° C.).

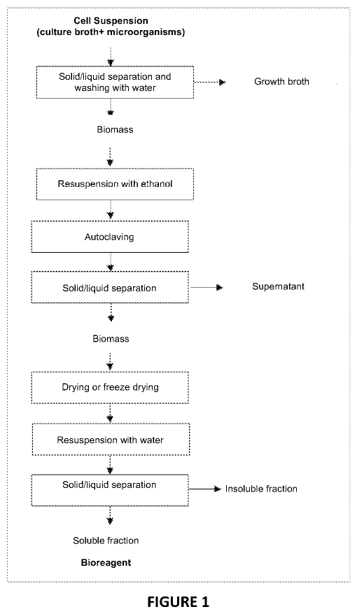

FIG. 1 illustrates a flowchart of the process for extracting bioreagent from microorganisms and comprises the phases of (i) solid/liquid separation and water washing; (ii) resuspension with ethanol; (iii) autoclaving; (iv) new solid/liquid separation; (v) drying or lyophilizing the biomass; (vi) resuspension with water (vii) new solid/liquid separation.

The solid/liquid separation phases may preferably be performed by centrifugation or filtration using a membrane with pores of 25 μm opening. Autoclaving should preferably be performed at a range of 0.5 to 1.5 pressure bar and temperature between 100 and 140° C.

The proportion of ethanol and water used in the process of extraction and dissolution of the soluble fraction, respectively, may be modified depending on the growth process of the microorganisms. Factors that can lead to process changes are: culture broth composition (can be replaced, for example, by tailings from the food industry), equipment and growing conditions (use, for example, biofermenters, immobilized cell inoculation).

In accordance with this invention, extraction of bioreagent from Rhodococcus bacteria (opacus, erythropolis) may include a purification phase.

The resulting bioreagent should preferably be stored for a maximum of 5 days at 4° C. for later use in froth flotation processes. The extraction method used allows the recovery of components associated with both intracellular compounds and those present in the cell wall of the microorganism. These substances are responsible for conferring hydrophobicity to the mineral surface.

Bioreagents extracted from Gram positive bacteria belonging to the genus Rhodococcus (species opacus, erythropolis) according to this invention may be used for flotation of any iron mineral, preferably hematite. It is also possible to float mineral systems, preferably the hematite-quartz system. However, flotation of ores containing other minerals of interest, such as calcite, dolomite and apatite, is also possible using the process of this invention.

According to this invention, the reagent to be added in the flotation phase may only comprise the bioreagent extracted from the bacteria Rhodococcus (opacus, erythropolis), in a concentration range of 25 to 200 mg/L, or may be used in conjunction with any of the following reagents, which are depressant reagent, collector and foaming reagent.

In accordance with this invention, the conditioning phase may be performed within a pH range of 3 to 7 for the hematite-quartz system.

Also according to this invention, the flotation phase can be performed in Hallimond tubes, flotation cells or flotation columns.

According to this invention, the flotation phase preferably consists of a direct flotation of the metal/element of interest.

Also according to this invention, the flotation phase may be performed within a pH range of 3 to 7 for the hematite-quartz system.

The results of froth flotation tests made with bioreagents, according to this invention, as shown in examples 4, 5 and 6, show the potential use of bioreagents as an alternative to synthetic reagents in mineral flotation processes. The use of bioreagents not only accelerates the flotation process, but increases the recovery of hematite, for example. FIG. 5 shows a composition of bar graphs comparing hematite floatability using R. opacus bacterium and its bioreagent for different pH values: (a) pH3, (b) pH5, (c) pH7, (d) pH9, (e) pH11.

The maximum floatability of hematite obtained using the bacterium (biomass) is 43% at neutral pH (FIG. 5 (c)) while the maximum recovery using bioreagent is 95% at acid pH (FIGS. 5 (a) and (b)). The high performance of bioreagent even in acidic environment is characteristic of most bioreagents that present stability even in environments with extreme temperature, pH and salinity conditions. The results showed the high affinity of the bioreagent of this invention with the hematite particles and a relatively low reagent consumption when compared to the use of bacteria (biomass).

### Example 1

Tests for extraction of bioreagent from microorganisms have been performed. The bacteria of the genus Rhodococcus (species opacus, erythropolis) used were obtained from the Brazilian Collection of Environment and Industry Microorganisms (CBMAI-UNICAMP).

The culture broth used for the growth of Rhodococcus opacus bacteria consisted of 10 g dm⁻³ glucose, 5 g dm⁻³ peptone, 3 g dm⁻³ malt extract, 3 g dm⁻³ yeast extract and 2 g dm⁻³ CaCO₃. The culture broth used for Rhodococcus erythropolis consisted of 17 g dm⁻³ casein extract, 3 g dm⁻³ soy flour, 5 g dm⁻³ NaCl, 2.5 g dm⁻³ glucose and 2 0.5 g dm⁻³ dipotassium phosphate. Bacteria were incubated in an orbital shaker at 125 rpm for 7 days.

After the growth period, the biomass from the growth broth (cell suspension) was separated by centrifugation at 4,000 rpm (FIG. 1). The biomass was washed with deionized water and centrifuged again to remove the remaining growth broth. Washing was repeated twice.

The biomass was resuspended using 500 mL of ethanol PA for each liter of cell suspension that fed the initial centrifugation process. For bioreagent extraction, the solution containing biomass and ethanol was autoclaved at 1 bar, 121° C. for 20 minutes.

After extraction, a further centrifugation step was performed to separate the biomass from the extracting solution. The supernatant was disposed of and the biomass dried in a kiln at 50° C. for 24 h.

The already dried biomass was resuspended in deionized water in the proportion of 125 mL of water for each liter of growth broth (cell suspension that fed the extraction process). The mixture was centrifuged and the water-insoluble fraction was disposed of while the soluble fraction was stored at 4° C. for a maximum of 5 days for use in microflotation and characterization assays.

### Example 2

In order to identify the functional groups present in the bioreagents obtained in Example 1, infrared analyzes (FT-IR) were performed using a Nicolet FTIR 2000 spectrometer and KBr matrix as a reference. The samples were dried at 50° C. and homogenized with KBr.

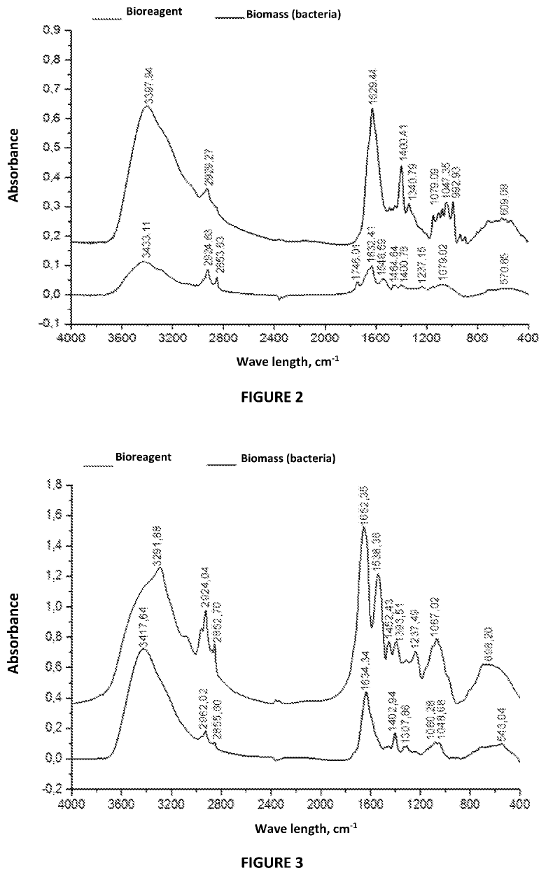

In order to compare the characteristics of bioreagents with those of microorganisms themselves (biomass), analyzes were performed under the same conditions as above for R. opacus and R. erythropolis bacteria, as shown in FIG. 2 and FIG. 3.

The infrared spectra of bacteria (biomass) show that the region below 1500 cm⁻¹ has a large number of adsorption peaks due to the variety of C—C, C—O and C—N bonds that may occur; this region is unique for each substance. In addition, an intense peak between 1750 and 1620 cm⁻¹ characteristic of aromatic compounds, aldehydes, ketones and esters was found. Mycolic acids, which form part of the cell casing and are responsible for the hydrophobicity of the bacteria, may be reflected by the peaks of the alkane, ketone and aldehyde groups. The presence of amino groups and aromatic compounds, which may be part of aromatic amino acids, indicate protein substances that play a determining role in flotation and flocculation processes.

With respect to bioreagent, the possible functional groups found in FT-IR analyzes are shown in Table 1. The alcohol, alkane, alkene and ketone groups found in the regions between 3417-3398, 2929-2855 and 1634-1629 cm⁻¹, respectively, may indicate the presence of mycolic acids. Identification of aromatic, as well as amino groups at wavelengths 1400, 1548 and 3350 cm⁻¹ may indicate the presence of polar amino acids such as tyrosine.

According to the literature, the proteins present in bacteria and their bioproducts may be responsible for flocculation flotation processes due to their amphiphilic character.

TABLE 1
Possible functional groups identified in the infrared spectroscopy
analysis of crude bioreagents.

| | | Possible | |
|---|---|---|---|
| Wavelength | | functional | |
| (cm⁻¹) | Intensity | grouping | Structure |
| 3397.94-3417 | High | Alcohols | 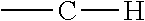 |
| 3350.00 | Average | Amines | 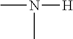 |
| 2929.27-2855 | Average | Alkanes | 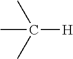 |
| 1629.44-1634 | High | Alkenes, Ketones | 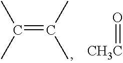 |
| 1400 | Average | Aromatic | 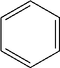 |
| 1047.35 | Average | Alkanes | 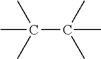 |

### Example 3

In order to verify another important feature of the bioreagents obtained in Example 1, the effect of bioreagents on the surface tension of distilled water at neutral pH was evaluated by varying the bioreagent concentration from 0 to 250 ppm. Surface tension measurements were performed by the ring method on a Kruss K10 digital tensiometer. In order to estimate the critical micellar concentration (CMC), two tangents were constructed at the minimum and maximum surface tension points, the intersection point of these lines indicate the CMC. For the bioreagent from R. opacus bacterium (RoBR) the CMC was 92 ppm, while for the bioreagent extracted from R. erythropolis bacterium (ReBR), 62 ppm.

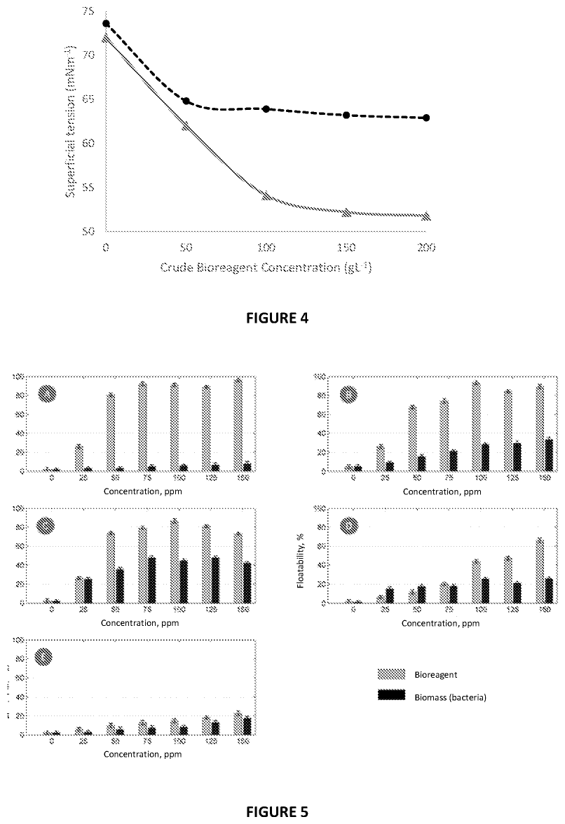

FIG. 4 shows the surface tension as a function of bioreagent concentration. Surface tension decreases to 50.5 mN m⁻¹ when using RoBR and 62 mN m⁻¹ when using ReBR. Bioreagents may be composed of polymeric substances that do not necessarily reduce surface tension, but may be effective in reducing interfacial tension between immiscible liquids and forming stable emulsions.

### Example 4

In order to verify hematite floatability, microflotation tests were performed according to this invention using modified Hallimond tube with 10⁻³ mol L⁻¹ NaCl as indifferent electrolyte, air flow 35 dm³ min⁻¹, particle size fraction +75-150 μm, conditioning time 2 minutes and flotation time 1 minute. Bioreagents concentration was varied from 25 to 150 ppm and pH from 3 to 11. Floatability was calculated as the ratio of floated mass to total mineral mass.

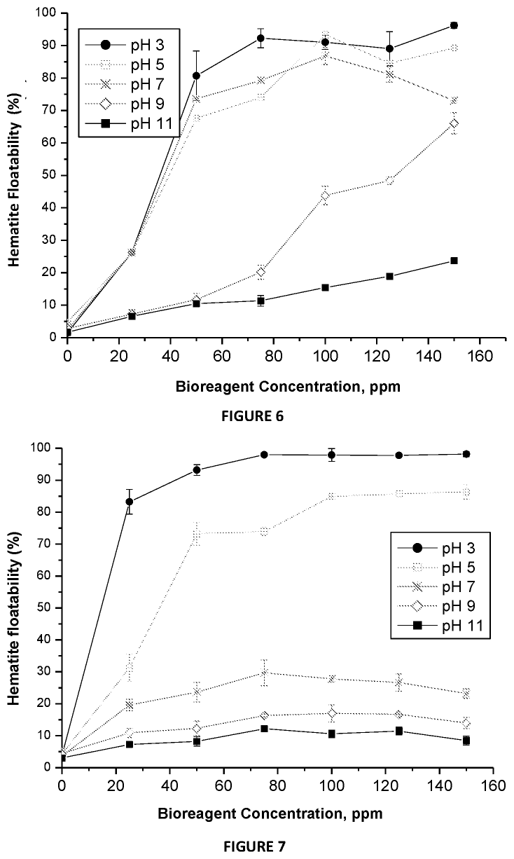

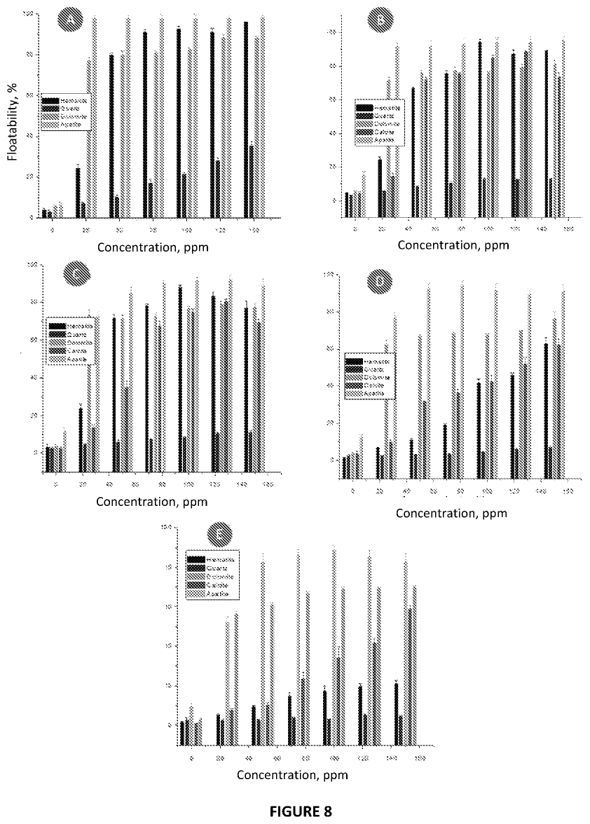

FIGS. 6 and 7 show the floatability of hematite using RoBR and ReBR, respectively. Both bioreagents have similar behavior: the maximum floatability (approximately 90%) of hematite occurred at pH 3 with 75 ppm bioreagent concentration. However, it was found that in the presence of RoBR, hematite can be float at acidic and neutral pH, while in the presence of ReBR, hematite flotation occurs only at acidic pH. The literature suggests that most non-toxic bioreagents are anionic. In addition, the isoelectric point of hematite occurs around 5.1. In this way, it is possible to correlate the pH of the medium to the bioreagent absorption on the mineral surface. In acid medium, there will be electrostatic attraction between the mineral surface and the anionic bioreagent resulting in a maximum adsorption and consequently in the maximum recovery of hematite. On the other hand, in basic medium, bioreagent absorption on the mineral surface will be minimal due to electrostatic repulsion.

### Example 5

In order to verify the floatability regions of calcite, dolomite, apatite, quartz and hematite, tests were performed under the same conditions as described in Example 4. The tests were performed with pure minerals.

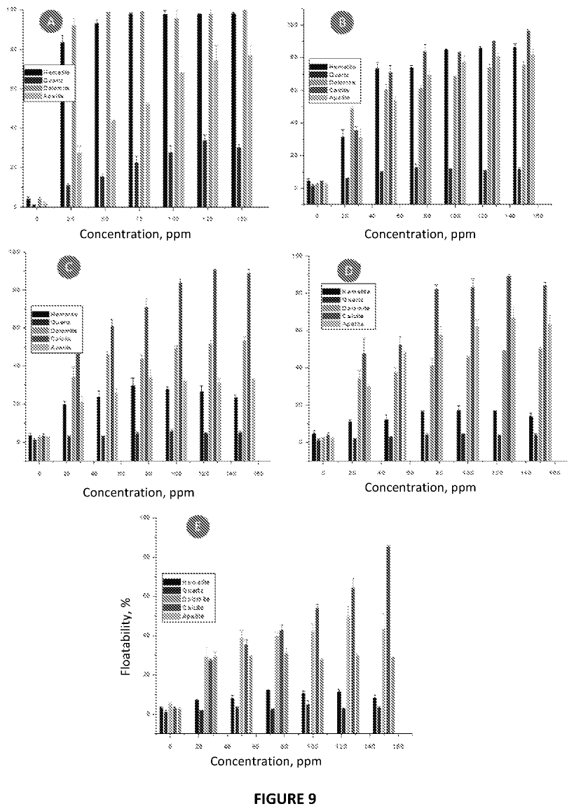

FIGS. 8 and 9 show bar graph compositions comparing the floatability of the different minerals above using both bioreagents (ReBR and RoBR). It is possible to observe several regions (windows) of selectivity among the studied minerals, for example:

a) Considering an ore composed by the hematite and quartz minerals, it can be verified that at pH 3, 5 and 7 it is possible to perform the direct flotation of hematite using between 50 and 150 ppm of RoBR. For the ReBR, this statement is only true for pH 3 and 5. At pH 3, the bioreagent concentration may be even lower, 25 ppm.
b) Considering a mineral composed by the minerals apatite and calcite, it is verified that at pH values 5, 7 and 9 it is possible to perform the direct flotation of the apatite using 25 ppm RoBR; and at pH 11 using 50 ppm RoBR. With ReBR, the separation between apatite and calcite can be performed at pH 7, through the direct flotation of calcite using between 100 and 150 ppm.
c) Considering a mineral composed by the minerals apatite and dolomite, it is verified that at pH values 3 it is possible to perform the direct flotation of the dolomite, when in presence of 25 ppm ReBR.

### Example 6

The hematite-quartz system was studied using the same procedure and flotation conditions listed in Example 5. The pH was maintained at 3 and three different hematite-quartz ratios (25H-75Q; 50H-50Q; 75H-25Q) were tested and two concentrations of ReBR (50 mg L⁻¹ and 100 mg L⁻¹). The results are presented on Table 2.

TABLE 2
Results of microflotation tests for the hematite-quartz system

| | | Mass | Mineral | | | Metallurgical |
|---|---|---|---|---|---|---|
| BR | H: Q | recovery | recovery (%) | | | recovery (%) |
| (mg/L) | (%) | (%) | Hematite | Quartz | FeT (%) | Fe |
| 50 | 25- | 32.40 | 57.97 | 42.03 | 38.85 | 55.5 |
| | 75 | | | | | |
| 100 | 25- | 37.58 | 59.01 | 40.99 | 39.55 | 56.5 |
| | 75 | | | | | |
| 50 | 50- | 52.61 | 87.33 | 12.67 | 58.53 | 83.6 |
| | 50 | | | | | |
| 100 | 50- | 56.62 | 89.32 | 10.68 | 59.86 | 85.5 |
| | 50 | | | | | |
| 50 | 75- | 69.87 | 93.66 | 6.34 | 62.77 | 89.7 |
| | 25 | | | | | |
| 100 | 75- | 71.82 | 95.31 | 4.69 | 63.88 | 91.3 |
| | 25 | | | | | |

The results showed that metallurgical recovery was similar for both concentrations of bioreagents studied when comparing the same mineral systems. For the 25% hematite-75% quartz ratio, the difference in metallurgical recovery was 1% (55.5 and 56.5% recovery for 50 and 100 mg L⁻¹ bioreagent, respectively). For the 50% hematite-50% quartz ratio, the Fe recovery was 83.6 and 85.5% when used 50 and 100 mg L⁻¹ bioreagent, respectively. For the 75% hematite-25% quartz ratio, the metallurgical recovery was 89.7 and 91.3% for 50 and 100 mg L⁻¹ bioreagent, respectively.

The same behavior can be seen when comparing mass recovery and iron content in the (float) concentrate. The use of double bioreagent (100 mg L⁻¹) showed little interference in the results of the above-mentioned flotation process. This effect can be attributed to the efficiency of RoBR during the bioflotation process.

1. Mineral beneficiation method, characterized by the fact that it uses bioreagent extracted from the bacterium Rhodococcus (opacus, erythropolis), which is subjected to bacterial growth, and comprises the following steps:
A—Comminution of ore and preparation of pulp;
B—Addition of reagents and conditioning;
C—Froth flotation.

2. Mineral beneficiation method, according to claim 1, characterized by the fact that said minerals include hematite, calcite, dolomite and apatite, aiming at the recovery of the metal/element of interest from an ore containing any of the minerals mentioned above.

3. Mineral beneficiation method, according to claim 1, characterized by the fact that said minerals include mineral systems, preferably the hematite-quartz system.

4. Mineral beneficiation method, according to claim 1, characterized by the fact that the extraction of bioreagent from the cell wall of Rhodococcus bacterium (opacus, erythropolis) is carried out by a solvent extraction process, preferably hot ethanol extraction (100-140° C.).

5. Mineral beneficiation method, according to claim 1, characterized by the fact that the reagent added in step B only includes the bioreagent extracted from the Rhodococcus bacterium (opacus, erythropolis) in a concentration range of 25 to 200 mg/L.

6. Mineral beneficiation method, according to claim 1, characterized by the fact that the reagents added in step B include the bioreagent extracted from the Rhodococcus bacterium (opacus, erythropolis), a depressant reagent, a collecting reagent and a foaming reagent.

7. Mineral beneficiation method, according to claim 1, characterized by the fact that the conditioning of step B is carried out in a pH range of 3 to 7 for the hematite-quartz system.

8. Mineral beneficiation method, according to claim 1, characterized by the fact that the froth flotation of step C can be performed in Hallimond tubes, flotation cells or flotation columns.

9. Mineral beneficiation method, according to claim 1, characterized by the fact that the froth flotation of step C is preferably direct flotation of the metal/element of interest.

10. Mineral beneficiation method, according to claim 1, characterized by the fact that the froth flotation of step C is carried out in a pH range of 3 to 7 for the hematite-quartz system.
